SEQUENCE LISTING (10 sequences)

SEQ ID NO: 1 - DNA, 26 bp, Artificial
  Hpx short primer set 1
acccctcttcctccgactagtgccca

SEQ ID NO: 2 - DNA, 26 bp, Artificial
  Hpx short primer set 2
gtgagtgcagcccaggagactggtca

SEQ ID NO: 3 - DNA, 72 bp, Artificial
  Hpx long primer set 1
agatatacatatgcaccatcaccatcatcacgacgatgacgacaagacccctcttcctccgactagtgccca

SEQ ID NO: 4 - DNA, 55 bp, Artificial
  Hpx long primer set 2
caaaaagtcgactcagcgggatggtcgggaagtgagtgcagcccaggagactggt

SEQ ID NO: 5 - protein, 630 aa, Artificial
  Hpx-hGH
TPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTHFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 6 - protein, 630 aa, Artificial
  hGH-Hpx
FPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFTPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 7 - protein, 630 aa, Artificial
  Hpx(H213A)-hGH
TPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGAGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTHFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 8 - protein, 630 aa, Artificial
  hGH-(H213A)Hpx
FPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFTPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGAGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 9 - protein, 641 aa, Artificial [sequence omitted]

SEQ ID NO: 10 - protein, 648 aa, Artificial [sequence omitted]

(19) United States
(12) Patent Application Publication
(10) Pub. No.: US 2009/0269843 A1
(43) Pub. Date: Oct. 29, 2009

(54) Hemopexin fusion proteins

(75) Inventors: Niels Blume, Beijing (CN); Jing Su, Beijing (CN); Ju Han, Beijing (CN); Lars Fogh Iversen, Holte (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/660,715

(22) PCT Filed: Aug. 16, 2005
(86) PCT No.: PCT/EP05/54015

§ 371 Date: Apr. 6, 2009

(30) Foreign Application Priority Data
Aug. 20, 2004 (DK) PA 2004 01259

(51) Int. Cl. C12N 5/10; C12N 15/63; A61K 38/16; C07K 16/18; C07K 19/00; C07H 21/00

(52) U.S. Cl. 435/348; 435/320.1; 514/12; 530/387.9; 530/394; 536/23.4

(57) ABSTRACT

Fusion proteins comprising a first protein fused to hemopexin are provided, said fusion proteins exhibit an increased circulation time.

### FIELD OF THE INVENTION

The invention relates to hemopexin (Hpx) fusion proteins. The invention also relates to methods of increasing the circulation time of proteins by fusing said proteins to Hpx or Hpx variants, and to therapeutic methods comprising the administration of Hpx fusion proteins to patients in need thereof.

### BACKGROUND OF THE INVENTION

Hemopexin (Hpx) is a 60 kDa protein which is present in the plasma in high amounts being second to only albumin, immuglobulins, and the plasma proteases. Hemopexin is often also referred to as beta-1B-glycoprotein. Hemopexin has a high affinity to heme (K_{D}<10⁻¹² M), and it is believed that the biological role of Hpx is related to the transportation of heme thus preventing heme induced oxidative damages and heme bound iron loss. Hemopexin sequesters free heme from the plasma which induces a structural change whereby the heme-Hpx complex gains increased affinity to receptors in the liver, where it is engulfed by receptor-mediated endocytosis and heme is released at the low pH in the endosomes. After that, Hpx is returned to circulation and can undergo further rounds of transportation.

Hemopexin is folded in two homologous domains, each of about 200 amino acids, joined by a 20 amino acid residue linker. There is ˜25% sequence identity between the two domains. Two histidines coordinate the heme iron, namely His213 from the linker peptide and His270 from a loop of the C-terminal domain, giving a stable bis-histidyl Fe(III) complex. The numbering is with respect to the mature protein. Clin. Chim. Acta, 312, 2001, 13-23 and DNA Cell Biol., 21, 2002, 297-304 provide recent reviews of Hpx chemistry.

Many attempts have been made to increase circulation time of proteins, and in particular of therapeutically relevant proteins. Classically, the protein has been conjugated to e.g. fatty acids, which is believed to bind to albumin, or to large molecules, such as PEG which increase molecular size to decrease renal clearance [J. Pharm. Sci., 86, 1365-1368, 1997; U.S. Pat. No. 4,179,337]. Another approach is to fuse the protein of interest to another protein, such as albumin [U.S. Pat. No. 5,045,312; WO 97/24445; WO 01/79271].

The number of known proteins with interesting biological or therapeutic activities is rapidly growing, inter alia as a result of the human genome project. However, the therapeutic potential of these novel proteins as well as “old” and well-established proteins is often limited by very short half-lifes in circulation. Hence, there remains a need for methods of increasing the half-life of proteins in circulation.

### SUMMARY OF THE INVENTION

The present invention relates to Hpx fusion proteins, comprising a first protein fused to Hpx or a Hpx variant, optionally via a linker.

In another embodiment, the invention relates to a method of increasing circulation time of a first protein, the method comprising fusing said first protein to Hpx or a Hpx variant, optionally via a linker.

In another embodiment, the invention relates to variants of Hpx.

In another embodiment, the invention relates to nucleic acid constructs encoding the Hpx fusion proteins or the Hpx variants of the present invention, to vectors containing said constructs, to host cells transformed with said vectors, and to methods of making the Hpx fusion proteins or the Hpx variants of the present invention using said constructs, vectors or host cells.

In another embodiment, the invention relates to specific antibodies raised against the Hpx fusion proteins or the Hpx variants of the present invention.

In another embodiment, the present invention relates to the Hpx fusion proteins of the present invention for use in therapy.

In another embodiment, the present invention relates to pharmaceutical compositions comprising a Hpx fusion protein of the present invention.

In another embodiment, the present invention relates to therapeutic methods comprising the administration of therapeutically effective amounts of a Hpx fusion protein of the present invention to a patient in need thereof.

In another embodiment, the present invention relates to the use of a Hpx fusion protein of the present invention in the manufacture of medicaments.

In another embodiment, the present invention relates to transgenic organisms modified to contain nucleic acid constructs of the present invention and to express Hpx fusion proteins or Hpx variants of the present invention.

### DESCRIPTION OF THE DRAWINGS

FIG. 1: The vector (pNNC19) contains AMP resistance and a T7 driven IPTG inducible hGH gene. Substitution of the Nde1/Sal1 segment with the Nde1/Sal1 containing hemopexin PCR amplicon results in an expression vector containing His6-D4K-hemopexin-hGH (pNCC36).

FIG. 2: Expression of His6-D4K-Hpx-hGH in E. coli. Lane 1 MW marker: 97, 66, 45, 30, 14.4 kDa; lane 2 pNNC19 hGH control (24.65 kDa); lane 3 pNNC36 un-induced; lane 4 IPTG induced; lane 5 before sonication; lane 6 after sonication; lane 7 soluble protein; and lane 8 inclusion bodies.

FIG. 3: UV scan of flow-through (a) and elution (b) from purification of His6-D4K-Hpx-hGH.

### DEFINITIONS

In the present context, “Hpx” is intended to indicate human Hpx, the sequence of which is available e.g. from Takahashi, Proc. Natl. Acad. Sci. U.S.A., 82(1), 73-77, 1985.

In the present context “a” is intended to indicate “one or more”.

In the present context “Hpx fusion protein” is intended to indicate a protein formed by the fusion of a first protein, e.g. a therapeutic protein, to a second protein which is Hpx or a Hpx variant. Said second protein is referred to as the “Hpx part” of the fusion protein. It is to be understood that said fusion may be direct in the sense that the C-terminal or N-terminal of Hpx or the Hpx variant is bound directly to the C-terminal or N-terminal of the first protein. The fusion may also be via a linker, wherein said linker at one end is bonded to Hpx or the Hpx variant, and at the other end is bonded to the above first protein. The point of attachment in the first protein and in the Hpx or the Hpx variant may be at any of the amino acid residues constituting said proteins, i.e. the C-terminal, the N-terminal or any of the amino acid residues in between.

In the present context, a “linker” is a moiety which serves to connect the two parts of the fusion protein, i.e. the Hpx part and the first protein. In one embodiment said linker is a bi-radical derived from a straight or branched C₁₋₅₀-alkan, straight or branched C₂₋₅₀-alken, straight or branched C₂₋₅₀-alkyn, a 1 to 50-membered straight or branched chain comprising carbon and at least one N, O or S atom in the chain, C₃₋₈cycloalkan, a 3 to 8-membered cyclic ring, aromatic or non-aromatic, comprising carbon and at least one N, O or S atom in the ring, benzene, naphthalene, or an amino acid, the structures optionally substituted with one or more of the following groups: Hydroxy, phenyl, phenoxy, benzyl, thienyl, oxo, amino, C₁₋₄-alkyl, —CONH₂, —CSNH₂, C₁₋₄ monoalkylamino, C₁₋₄ dialkylamino, acylamino, sulfonyl, carboxy, carboxamido, halogen, C₁₋₆ alkoxy, C₁₋₆alkylthio, trifluoroalkoxy, alkoxycarbonyl, and haloalkyl. In another embodiment, said linker represents a protein diradical comprising up to 50 amino acid residues, such as up to 40, 30, 20 or 10 amino acid residues. It may be desirable to cleave the Hpx fusion protein at some point in which case a cleavage site, e.g. for enzymatic hydrolysis may be comprised in the linker.

In the present context, the term “protein” is intended to indicate a sequence of 2 or more amino acids bonded by peptide bonds. Preferably, a protein comprises more than 20 amino acid residues, such as more than 40, such as more than 50, wherein said amino acids may be natural or unnatural. It is to be understood that the term also is intended to include proteins which have been further derivatized, e.g. by the attachment of lipophilic or PEG groups. In one embodiment, a protein is intended to indicate a therapeutic protein.

A “therapeutically effective amount” of a compound as used herein means an amount sufficient to cure, alleviate or partially arrest the clinical manifestations of a given disease and its complications. An amount adequate to accomplish this is defined as “therapeutically effective amount”. Effective amounts for each purpose will depend e.g. on the severity of the disease or injury as well as the weight, sex, age, the effect to be achieved and general state of the subject to be treated. It will be understood that determining an appropriate dosage may be obtained using routine experimentation, e.g. by constructing a matrix of values and testing different points in the matrix, which is all within the ordinary skills of a trained physician or veterinary.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. Nevertheless, it is to be understood that treatment and prophylaxis are separate embodiments of the invention. The patient to be treated is preferably a mammal, in particular a human being, but it may also include animals, such as dogs, cats, cows, sheep and pigs.

The term “therapeutic protein” is intended to indicate a protein having one or more therapeutic and/or biological activities in vivo. A therapeutic protein is useful to treat or ameliorate a disease, condition or disorder. Although the activity of therapeutic proteins ultimately is to be effected in vivo, there are many in vitro assays known to the person skilled in the art whereby therapeutic activity can be measured.

### DESCRIPTION OF THE INVENTION

In one embodiment, the invention relates to Hpx fusion proteins, comprising a first protein fused to Hpx or a Hpx variant, optionally via a linker, wherein said fusion protein has an increased circulation time compared to said first protein. In particular, increased circulation time is intended to indicate increased circulation time in man.

In one embodiment, said first protein is a therapeutic protein.

In one embodiment, said first protein comprises at least 30 amino acid residues.

In the present context, a “Hpx variant” is understood to be a variant which when fused to a first protein extends the circulation time of said first protein. In general terms, a “variant” of a protein is obtained by substituting one or more amino acid residues with another natural or unnatural amino acid; by adding one or more natural or unnatural amino acids; and/or by deleting one or more amino acid residue from said protein, optionally followed by further derivatizing of one or more amino acid residue. In a particular embodiment, up to 40, such as up to 30, such as up to 20, such as up to 10, such as up to 5 amino acid residues have been substituted, added and/or deleted. In particular, such substitutions are conservative in the sense that one amino acid residue is substituted by another amino acid residue from the same group, i.e. by another amino acid residue with similar properties. Amino acids may conveniently be divided in the following groups based on their properties: Basic amino acids (such as arginine, tyrosine, lysine, histidine), acidic amino acids (such as glutamic acid and aspartic acid), polar amino acids (such as glutamine and asparagine), hydrophobic amino acids (such as leucine, isoleucine, valine), aromatic amino acids (such as phenylalanine, tryptophan, tyrosine) and small amino acids (such as glycine, alanine, serine, threonine, methionine).

Human hemopexin consists of 439 amino acid residues, and Hpx variants include the protein obtained by deletions from the N-terminal of the full length Hpx. Hemopexin variants obtained by N-terminal deletions may be described as Hpx₁₋ₙ, wherein n is an integer from 339 to 438. In this nomenclature “Hpx₁₋₄₀₀” is intended to indicate the Hpx variant consisting amino acid residues 1-400, i.e. wherein amino acid residue 401-439 has been deleted. In particular, n is an integer from 360 to 438, such as from 400 to 438, such as from 420 to 438. Hemopexin variants also include the protein obtained by deletions from the C-terminal of the full length Hpx. Hemopexin variants obtained by C-terminal deletions may be described as Hpxₘ₋₄₃₉, wherein m is an integer from 2 to 100. In this nomenclature, “Hpx₅₀₋₄₃₉” is intended to indicate the Hpx variant consisting of amino acid residues 50 to 439, i.e. wherein amino acid residue 1 to 49 has been deleted. In particular, m is an integer from 2-20, 2-50 or 2-80. Hemopexin variants also include the combination of C-terminal and N-terminal deletions, which according to the above nomenclature can be described as Hpx_{q-p}, wherein q is an integer from 2 to 50, and p is an integer from 389 to 438. In particular, q is an integer from 2-10, 2-20 or 2-30 in combination with p being any of an integer from 389-438, 410-438 or 420-438.

In another embodiment, Hpx variants of the present invention have at least 80%, such as at least 85%, such as at least 90%, such as at least 95% identity with Hpx.

The term “identity” as known in the art, refers to a relationship between the sequences of two or more proteins, as determined by comparing the sequences. In the art, “identity” also means the degree of sequence relatedness between proteins, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related proteins can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math., 48:1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res., 12:387 (1984); Genetics Computer Group, University of Wisconsin, Madison, Wis.), BLASTP, BLASTN, and FASTA (Altschul et al., J. Mol. Biol., 215:403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two proteins for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp. 3 (1978) for the PAM 250 comparison matrix; Henikoff et al., Proc. Natl. Acad. Sci. USA, 89:10915-10919 (1992) for the BLOSUM 62 comparison matrix) is also used by the algorithm.

Preferred parameters for a protein sequence comparison include the following:

Algorithm: Needleman et al., J. Mol. Biol, 48:443-453 (1970); Comparison matrix: BLOSUM 62 from Henikoff et al., Proc. Natl. Acad. Sci. USA, 89:10915-10919 (1992); Gap Penalty: 12, Gap Length Penalty: 4, Threshold of Similarity: 0.

The GAP program is useful with the above parameters. The aforementioned parameters are the default parameters for protein comparisons (along with no penalty for end gaps) using the GAP algorithm.

As discussed above, the binding of heme to Hpx gives rise to a conformational change which increases the affinity to receptors in the liver. It is believed that a Hpx fusion protein comprising a Hpx variant in which the heme binding capacity has been reduced or even deleted will exhibit a further increase in circulation time as the affinity to liver receptors has been lost. Binding of heme takes place at His 213 and His 270 (numbering with respect to mature protein), and point mutations as these sites will remove the heme binding affinity of Hpx, and thus the liver receptor affinity. In on embodiment, Hpx variants include Hpx wherein one or both of His 213 and His 270 have been deleted or substituted with another amino acid so that the heme binding capacity of Hpx is reduced by at least 20%, such as at least 50%, such as at least 80%. Examples of such other amino acids include Ala and Cys.

Renal clearance which is one mechanism for clearing proteins from circulation is largely determined by the size of the molecules to be cleared, and the size of a molecule again largely depends on its molecular weight. The cut-off value of the kidneys is approximately 70 kDa, but this is also dependent on the charge of the molecule, i.e. the kidneys have a higher cut-off value for uncharged molecules than for charged molecules. In one embodiment, the invention relates to Hpx fusion proteins with a molecular weight above 60 kDa, such as above 70 kDa, such as above 80 kDa.

In one embodiment, a linker made of up to 30 amino acid residues is inserted between the first protein and the Hpx part. In particular, this linker may be made of up to 25 amino acid residues, such as up to 20 amino acid residues, such as up to 15 amino acid residues, such as up to 10 amino acid residues, such as up to 5 amino acid residues, such as 1, 2, 3 or 4 amino acid residues.

In one embodiment, the present invention relates to Hpx variants as indicated above.

In one embodiment, the invention provides a method of increasing the circulation time, and in particular the circulation time in man, of a first protein, the method comprising fusing said first protein to Hpx or a Hpx variant. In particular, such method may further comprise a step in which the fusion protein is assayed in an suitable assay to assess the change in circulation time effected. In particular, such method may further comprise a step wherein the Hpx fusion protein is formulated in a pharmaceutical composition.

An increase in circulation time may be quantified as a decrease in clearance (CL) or as an increase in mean residence time (MRT). Fusion proteins of the present invention for which the CL is decreased to 50% or less of the CL of the first protein as determined in a suitable assay is said to have an increased circulation time. Fusion proteins of the present invention for which MRT is increased to 150% or more of the MRT of the first protein in a suitable assay is said to have an increased circulation time. Clearance and mean residence time can be assessed in standard pharmacokinetic studies using suitable test animals. It is within the capabilities of a person skilled in the art to choose a suitable test animal for a given fusion protein. Tests in human, of course, represent the ultimate test.

If, for example, the fusion protein comprises a growth hormone, then examples of suitable test animals include normal, Sprague-Dawley male rats, mice and cynomolgus monkeys. Typically the mice and rats are in injected in a single subcutaneous bolus, while monkeys may be injected in a single subcutaneous bolus or in a single iv dose. The amount injected depends on the test animal. Subsequently, blood samples are taken over a period of one to five days as appropriate for the assessment of CL and MRT. The blood samples are conveniently analysed by ELISA techniques.

In one embodiment, the fusion proteins of the present invention are selected from

Hpx-hGH (SEQ ID No 5);

hGH-Hpx (SEQ ID No 6);

Hpx(H213A)-hGH (SEQ ID No 7);

hGH-Hpx(H213A) (SEQ ID No 8);

His6-D4K-Hpx-hGH; (SEQ ID No 9) and

His 8-PTERP-D4K-hGH-Hpx (SEQ ID No 10).

Methionine is coded by start codon, and is therefore present in the N-terminal of proteins immediately after translation. However, the N-terminal methionine may by removed in post translation modifications; something which depends, e.g. on the neighboring amino acid residue. It may be difficult to predict whether or not the resulting protein will have an N-terminal methionine; often this has to be determined by N-terminal sequencing. Consequently, the invention also relates to the above mentioned proteins (SEQ ID No 5-10) with an additional N-terminal methionine.

Hemopexin fusion proteins and Hpx variant of the present invention can be used to raise antibodies that specifically bind to the proteins of the present invention. In the present context, “antibodies” include monoclonal and polyclonal antibodies, and antigen-binding fragments thereof, such as F(ab′)₂ and Fab fragments, including genetically engineered antibodies. Anti-bodies are said to be specific if they bind to a peptide of the present invention with a Kₐ greater than or equal to 10⁷ M⁻¹. Methods for preparing antibodies are disclosed in e.g. Hurrell J. G. R. (Ed.) Monoclonal Hybridoma Antibodies: Techniques and Applications, CRC Press, Boca Raton, Fla., 1982 and Sambrok, Molecular Cloning: A Laboratory Manual, Cold Spring Harbour, N.Y., 1989. Antibodies raised against peptides of the present invention may be used e.g. in diagnostic assays and for affinity purification.

In one embodiment, the invention provides nucleic acid constructs encoding proteins of the present invention.

The fusion proteins of the present invention may be prepared in a number of different ways. They may be synthesized using protein synthesis methods well-known to persons skilled in the art. It is also possible to express the first protein and the Hpx part separately in suitable hosts and fuse the two proteins subsequently, optionally via a linker. In a particular embodiment, however, the Hpx fusion protein is expressed as such in a suitable host after incorporation of a suitable nucleic acid construct into said host.

As used herein the term “nucleic acid construct” is intended to indicate any nucleic acid molecule of cDNA, genomic DNA, synthetic DNA or RNA origin. The term “construct” is intended to indicate a nucleic acid segment which may be single- or double-stranded, and which may be based on a complete or partial nucleotide sequence encoding a protein of interest. The construct may optionally contain other nucleic acid segments.

The nucleic acid construct of the invention encoding the protein of the invention may suitably be of genomic or cDNA origin, for instance obtained by preparing a genomic or cDNA library and screening for DNA sequences coding for all or part of the protein by hybridization using synthetic oligonucleotide probes in accordance with standard techniques (cf. Sambrook et al., supra). For the present purpose, the DNA sequence encoding the protein is preferably of human origin, i.e. derived from a human genomic DNA or cDNA library. In particular, the DNA sequence may be of human origin, e.g. cDNA from a particular human organ or cell type or a gene derived from human genomic DNA.

The nucleic acid construct of the invention encoding the protein may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g. in an automatic DNA synthesizer, purified, annealed, ligated and cloned in suitable vectors.

Furthermore, the nucleic acid construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleic acid construct, in accordance with standard techniques.

The nucleic acid construct may also be prepared by polymerase chain reaction using specific primers, for instance as described in U.S. Pat. No. 4,683,202 or Saiki et al., Science 239 (1988), 487-491.

In one embodiment, the nucleic acid construct is a DNA construct which term will be used exclusively in the following.

In one embodiment, the present invention relates to a recombinant vector comprising a DNA construct of the invention. The recombinant vector into which the DNA construct of the invention is inserted may be any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the protein of the invention is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the protein.

The promoter may be any DNA sequence which shows transcriptional activity in the host cell of choice and may be derived from genes encoding proteins either homologous or heterologous to the host cell.

Examples of suitable promoters for directing the transcription of the DNA encoding the protein of the invention in mammalian cells are the SV40 promoter (Subramani et al., Mol. Cell. Biol. 1 (1981), 854-864), the MT-1 (metallothionein gene) promoter (Palmiter et al., Science 222 (1983), 809-814) or the adenovirus 2 major late promoter.

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304 (1983), 652-654) promoters.

Examples of suitable promoters for use in filamentous fungus host cells are, for instance, the ADH3 promoter (McKnight et al., The EMBO J. 4 (1985), 2093-2099) or the tpiA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizomucor miehei lipase, A. oryzae alkaline protease, A. oryzae triose phosphate isomerase or A. nidulans acetamidase. Preferred are the TAKA-amylase and gluA promoters.

Examples of suitable promoters for use in bacterial host cells include the promoter of the Bacillus stearothermophilus maltogenic amylase gene, the Bacillus licheniformis alphaamylase gene, the Bacillus amyloliquefaciens BAN amylase gene, the Bacillus subtilis alkaline protease gen, or the Bacillus pumilus xylosidase gene, or by the phage Lambda P_{R} or P_{L} promoters or the E. coli lac, trp or tac promoters.

The DNA sequence encoding a protein of the invention may also, if necessary, be operably connected to a suitable terminator, such as the bacterial terminator T7, the human growth hormone terminator (Palmiter et al., op cit.) or (for fungal hosts) the TPI1 (Alber and Kawasaki, op cit.) or ADH3 (McKnight et al., op cit.) terminators. The vector may further comprise elements such as polyadenylation signals (e.g. from SV40 or the adenovirus 5 Elb region), transcriptional enhancer sequences (e.g. the SV40 enhancer) and translational enhancer sequences (e.g. the ones encoding adenovirus VA RNAs).

The recombinant vector of the invention may further comprise a DNA sequence enabling the vector to replicate in the host cell in question. An example of such a sequence (when the host cell is a mammalian cell) is the SV40 origin of replication.

When the host cell is a yeast cell, suitable sequences enabling the vector to replicate are the yeast plasmid 2μ replication genes REP 1-3 and origin of replication.

When the host cell is a bacterial cell, sequences enabling the vector to replicate are DNA polymerase III complex encoding genes and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For filamentous fungi, selectable markers include amdS, pyrG, argB, niaD and sC.

To direct a protein of the present invention into the secretory pathway of the host cells, a secretory signal sequence (also known as a leader sequence, prepro sequence or pre sequence) may be provided in the recombinant vector. The secretory signal sequence is joined to the DNA sequence encoding the protein in the correct reading frame. Secretory signal sequences are commonly positioned 5′ to the DNA sequence encoding the protein. The secretory signal sequence may be that which is normally associated with the protein or may be from a gene encoding another secreted protein.

For secretion from yeast cells, the secretory signal sequence may encode any signal peptide which ensures efficient direction of the expressed protein into the secretory pathway of the cell. The signal peptide may be naturally occurring signal peptide, or a functional part thereof, or it may be a synthetic peptide. Suitable signal peptides have been found to be the α-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and upstream of the DNA sequence encoding the protein. The function of the leader peptide is to allow the expressed protein to be directed from the endoplasmic reticulum to the Golgi apparatus and further to a secretory vesicle for secretion into the culture medium (i.e. exportation of the protein across the cell wall or at least through the cellular membrane into the periplasmic space of the yeast cell). The leader peptide may be the yeast α-factor leader (the use of which is described in e.g. U.S. Pat. No. 4,546,082, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

The procedures used to ligate the DNA sequences coding for the present protein, the promoter and optionally the terminator and/or secretory signal sequence, respectively, and to insert them into suitable vectors containing the information necessary for replication, are well known to persons skilled in the art (cf., for instance, Sambrook et al., op. cit.).

The host cell into which the DNA construct or the recombinant vector of the invention is introduced may be any cell which is capable of producing the present protein and includes bacteria, yeast, fungi and higher eukaryotic cells.

Examples of bacterial host cells which, on cultivation, are capable of producing the protein of the invention are grampositive bacteria such as strains of Bacillus, such as strains of B. subtilis, B. licheniformis, B. lentus, B. brevis, B. stearothermophilus, B. alkalophilus, B. amyloliquefaciens, B. coagulans, B. circulans, B. lautus, B. megatherium or B. thuringiensis, or strains of Streptomyces, such as S. lividans or S. murinus, or gram negative bacteria such as Echerichia coli. The transformation of the bacteria may be effected by protoplast trans-formation or by using competent cells in a manner known per se (cf. Sambrook et al., supra).

When expressing a protein in bacteria such as E. coli, the protein may be retained in the cytoplasm, typically as insoluble granules (known as inclusion bodies), or may be directed to the periplasmic space by a bacterial secretion sequence. In the former case, the cells are lysed and the granules are recovered and denatured after which the protein is refolded by diluting the denaturing agent. In the latter case, the protein may be recovered from the periplasmic space by disrupting the cells, e.g. by sonication or osmotic shock, to release the contents of the periplasmic space and recovering the protein.

Examples of suitable mammalian cell lines are the COS (ATCC CRL 1650), BHK (ATCC CRL 1632, ATCC CCL 10), CHL (ATCC CCL39) or CHO (ATCC CCL 61) cell lines. Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Graham and van der Eb, Virology 52 (1973), 456; and Neumann et al., EMBO J. 1 (1982), 841-845.

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous proteins therefrom are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequence encoding a protein of the invention may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277 and EP 230 023. The transformation of F. oxysporum may, for instance, be carried out as described by Malardier et al., 1989, Gene 78: 147-156.

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

Transformation of insect cells and production of heterologous proteins therein may be performed as described in U.S. Pat. No. 4,745,051; U.S. Pat. No. 4,879,236; U.S. Pat. Nos. 5,155,037; 5,162,222; EP 397,485) all of which are incorporated herein by reference. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

The transformed or transfected host cell described above is then cultured in a suitable nutrient medium under conditions permitting the expression of the present protein, after which the resulting protein is recovered from the culture.

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The protein produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gel filtration chromatography, affinity chromatography, or the like, dependent on the type of protein in question.

It is also within the scope of the present invention to employ transgenic animal technology to produce the present protein. A transgenic animal is one in whose genome a heterologous DNA sequence has been introduced. In particular, the protein of the invention may be expressed in the mammary glands of a non-human female mammal, in particular one which is known to produce large quantities of milk. Examples of preferred mammals are livestock animals such as goats, sheep and cattle, although smaller mammals such as mice, rabbits or rats may also be employed.

The DNA sequence encoding the present protein may be introduced into the animal by any one of the methods previously described for the purpose. For instance, to obtain expression in a mammary gland, a transcription promoter from a milk protein gene is used. Milk protein genes include the genes encoding casein (cf. U.S. Pat. No. 5,304,489), beta-lactoglobulin, alpha-lactalbumin and whey acidic protein. The currently preferred promoter is the beta-lactoglobulin promoter (cf. Whitelaw et al., Biochem J. 286, 1992, pp. 31-39).

It is generally recognized in the art that DNA sequences lacking introns are poorly expressed in transgenic animals in comparison with those containing introns (cf. Brinster et al., Proc. Natl. Acad. Sci. USA 85, 1988, pp. 836-840; Palmiter et al., Proc. Natl. Acad. Sci. USA 88, 1991, pp. 478-482; Whitelaw et al., Transgenic Res. 1, 1991, pp. 3-13; WO 89/01343; WO 91/02318). For expression in transgenic animals, it is therefore preferred, whenever possible, to use genomic sequences containing all or some of the native introns of the gene encoding the protein of interest. It may also be preferred to include at least some introns from, e.g. the beta-lactoglobulin gene. One such region is a DNA segment which provides for intron splicing and RNA polyadenylation from the 3′ non-coding region of the ovine beta-lactogloblin gene. When substituted for the native 3′ non-coding sequences of a gene, this segment may will enhance and stabilise expression levels of the protein of interest. It may also be possible to replace the region surrounding the initiation codon of the protein of interest with corresponding sequences of a milk protein gene. Such replacement provides a putative tissue-specific initiation environment to enhance expression.

For expression of the present protein in transgenic animals, a nucleotide sequence encoding the protein is operably linked to additional DNA sequences required for its expression to produce expression units. Such additional sequences include a promoter as indicated above, as well as sequences providing for termination of transcription and polyadenylation of mRNA. The expression unit further includes a DNA sequence encoding a secretory signal sequence operably linked to the sequence encoding the protein. The secretory signal sequence may be one native to the protein or may be that of another protein such as a milk protein (cf. von Heijne et al., Nucl. Acids Res. 14, 1986, pp. 4683-4690; and U.S. Pat. No. 4,873,316).

Construction of the expression unit for use in transgenic animals may conveniently be done by inserting a DNA sequence encoding the present protein into a vector containing the additional DNA sequences, although the expression unit may be constructed by essentially any sequence of ligations. It is particularly convenient to provide a vector containing a DNA sequence encoding a milk protein and to replace the coding region for the milk protein with a DNA sequence coding for the present protein, thereby creating a fusion which includes expression control sequences of the milk protein gene.

The expression unit is then introduced into fertilized ova or early-stage embryos of the selected host species. Introduction of heterologous DNA may be carried out in a number of ways, including microinjection (cf. U.S. Pat. No. 4,873,191), retroviral infection (cf. Jaenisch, Science 240, 1988, pp. 1468-1474) or site-directed integration using embryonic stem cells (reviewed by Bradley et al., Bio/Technology 10, 1992, pp. 534-539). The ova are then implanted into the oviducts or uteri of pseudopregnant females and allowed to develop to term. Offspring carrying the introduced DNA in their germ line can pass the DNA on to their progeny, allowing the development of transgenic herds.

General procedures for producing transgenic animals are known in the art, cf. for instance, Hogan et al., Manipulating the Mouse Embryo: A Laboratory Manual, Cold Spring Harbor Laboratory, 1986; Simons et al., Bio/Technology 6, 1988, pp. 179-183; Wall et al., Biol. Reprod. 32, 1985, pp. 645-651; Buhler et al., Bio/Technology 8, 1990, pp. 140-143; Ebert et al., Bio/Technology 6: 179-183, 1988; Krimpenfort et al., Bio/Technology 9: 844-847, 1991, Wall et al., J. Cell. Biochem. 49:113-120, 1992; U.S. Pat. No. 4,873,191, U.S. Pat. No. 4,873,316; WO 88/00239, WO 90/05188; WO 92/11757 and GB 87/00458. Techniques for introducing heterologous DNA sequences into mammals and their germ cells were originally developed in the mouse. See, e.g. Gordon et al., Proc. Natl. Acad. Sci. USA 77: 7380-7384, 1980, Gordon and Ruddle, Science 214: 1244-1246, 1981; Palmiter and Brinster, Cell 41: 343-345, 1985; Brinster et al., Proc. Natl. Acad. Sci. USA 82: 4438-4442, 1985; and Hogan et al. (ibid.). These techniques were subsequently adapted for use with larger animals, including livestock species (see e.g., WO 88/00239, WO 90/01588 and WO 92/11757; and Simons et al., Bio/Technology 6: 179-183, 1988). To summarize, in the most efficient route used to date in the generation of transgenic mice or livestock, several hundred linear molecules of the DNA of interest are injected into one of the pro-nuclei of a fertilized egg according to techniques which have become standard in the art. Injection of DNA into the cytoplasm of a zygote can also be employed.

In another embodiment, the first protein and Hpx part are expressed separately. The first protein may then be reacted with a bi-functional linker (activation) whereby the linker is bonded to the first protein via a first functional group. The activated protein is subsequently reacted with the Hpx part whereby the Hpx part is bonded to the linker via the second functional group. It is clear to the person skilled in the art that the reaction order may be reversed so that the linker is first reacted with the Hpx part followed by a reaction with the first protein.

Numerous functional groups have been disclosed in the literature which are useful for forming a bond between a protein and a linker. Relevant references are, e.g. WO 03/044056 and Biomaterials, 22, 405-417, 2001. Typically, groups in proteins which are useful points of attachments are amines, hydroxyls, thiols, aldehydes and ketones, which may be present in the native protein or which may be generated, e.g. by oxidation. When the first protein and the Hpx part are fused via a linker, the linker may in principle be attached to any amino acid residue in the first protein and to any amino acid residue in the Hpx part.

The first protein may be further derivatised, e.g. by attachment of lipophilic or PEG groups to further modify the properties. Also, upon fusion to Hpx or a Hpx variant, the resulting fused protein may be further derivatized, e.g. by attachment of lipophilic or PEG groups to further modify the properties of the fused protein.

Any protein may in principle be fused to Hpx or a Hpx variant according to the present invention. Such proteins include enzymes, peptide hormones, growth factors, antibodies, cytokines, receptors, lymphokines, and vaccines antigens, and particular mentioning is made of therapeutic proteins, such as insulin, glucagon-like peptide 1 (GLP-1), glucagons-like peptide 2 (GLP-2), growth hormone, cytokines, trefoil factor peptides (TTF), peptide melanocortin receptor modifiers and Factor VII compounds. In one embodiment, the invention relates to Hpx fusion proteins which in addition the fusion of the Hpx or Hpx variant is also derivatized, e.g. with lipophilic groups or PEG to obtain a further modification of the properties of the protein.

Particular applicable insulin is human insulin. In the present context the term “human insulin” refers to naturally produced insulin or recombinantly produced insulin. Recombinant human insulin may be produced in any suitable host cell, for example the host cells may be bacterial, fungal (including yeast), insect, animal or plant cells. Many insulin compounds have been disclosed in the literature, and they too are particular useful in the methods of the present invention. By “insulin compound” (and related expressions) is meant human insulin in which one or more amino acids have been deleted and/or replaced by other amino acids, natural or unnatural, and/or human insulin comprising additional amino acids, natural or unnatural, i.e. more than 51 amino acids, and/or human insulin in which at least one organic substituent is bound to one or more of the amino acids.

Examples of GLP-1 relevant to the present invention include human GLP-1 and GLP-1 compounds. Human GLP-1 is a 37 amino acid residue peptide originating from preproglucagon which is synthesised i.a. in the L-cells in the distal ileum, in the pancreas and in the brain. GLP-1 is an important gut hormone with regulatory function in glucose metabolism and gastrointestinal secretion and metabolism. Processing of preproglucagon to give GLP-1 (7-36)-amide, GLP-1 (7-37) and GLP-2 occurs mainly in the L-cells. The fragments GLP-1 (7-36)-amide and GLP-1 (7-37) are both glucose-dependent insulinotropic agents. In the past decades a number of structural analogues of GLP-1 were isolated from the venom of the Gila monster lizards (Heloderma suspectum and Heloderma horridum). Exendin-4 is a 39 amino acid residue peptide isolated from the venom of Heloderma horridum, and this peptide shares 52% homology with GLP-1. Exendin-4 is a potent GLP-1 receptor agonist which has been shown to stimulate insulin release and ensuring lowering of the blood glucose level when injected into dogs. The group of GLP-1(1-37) and exendin-4(1-39) and certain fragments, analogues and derivatives thereof (designated GLP-1 compounds herein) are potent insulinotropic agents, and they are all applicable in the method of the present invention. Insulinotropic fragments of GLP-1 (1-37) are insulinotropic peptides for which the entire sequence can be found in the sequence of GLP-1 (1-37) and where at least one terminal amino acid has been deleted. Examples of insulinotropic fragments of GLP-1 (1-37) are GLP-1 (7-37) wherein the amino acid residues in positions 1-6 of GLP-1 (1-37) have been deleted, and GLP-1 (7-36) where the amino acid residues in position 1-6 and 37 of GLP-1 (1-37) have been deleted. Examples of insulinotropic fragments of exendin-4(1-39) are exendin-4(1-38) and exendin-4(1-31). The insulinotropic property of a compound may be determined by in vivo or in vitro assays well known in the art. For instance, the compound may be administered to an animal and monitoring the insulin concentration over time. Insulinotropic analogs of GLP-1 (1-37) and exendin-4(1-39) refer to the respective molecules wherein one or more of the amino acids residues have been exchanged with other amino acid residues, natural or unnatural, and/or from which one or more amino acid residues have been deleted and/or from which one or more amino acid residues, natural or unnatural, have been added with the proviso that said analogue either is insulinotropic or is a prodrug of an insulinotropic compound.

GLP-2 and GLP-2 compounds may also be modified according to the present invention. In the present context a GLP-2 compound binds to a GLP-2 receptor, preferably with an affinity constant (K_{D}) or a potency (EC₅₀) of below 1 μM, e.g. below 100 nM. The term “GLP-2 compound” is intended to indicate human GLP-2 in which one or more amino acid residue has been deleted and/or replaced by another amino acid residue, natural or unnatural, and/or human GLP-2 comprising additional amino acid residues, and/or human GLP-2 in which at least one organic substituent is bound to one or more of the amino acid residues. In particular, those peptides are considered, which amino acid sequence exhibit at any sequence of 33 consecutive amino acids more than 60% of the amino acid sequence of human GLP-2. Also those peptides are considered, which amino acid sequence exhibit at any sequence of 37 consecutive amino acids more than 60% of the amino acid sequence of human GLP-2 when up to four amino acids are deleted from the amino acid sequence. Also those peptides are considered, which amino acid sequence exhibit at any sequence of 31 consecutive amino acids more than 60% of the amino acid sequence of GLP-2, when up to two amino acids are added to their amino acid sequence. The term “GLP compounds” also includes natural allelic variations that may exist and occur from one individual to another. Also, degree and location of glycosylation or other post-translation modifications may vary depending on the chosen host cells and the nature of the host cellular environment. Candidate GLP-2 compounds, which may be used according to the present invention include the GLP-2 compounds described in WO 96/32414, WO 97/39031, WO 98/03547, WO 96/29342, WO 97/31943, WO 98/08872, which are all incorporated herein by reference.

Factor VII compounds relevant to the present invention encompass wild-type Factor VII (i.e., a protein having the amino acid sequence disclosed in U.S. Pat. No. 4,784,950), as well as variants of Factor VII exhibiting substantially the same, reduced or improved biological activity relative to wild-type Factor VII, Factor VII-related polypeptides as well as Factor VII derivatives and Factor VII conjugates. The term “Factor VII compounds” is intended to encompass Factor VII polypeptides in their uncleaved (zymogen) form, as well as those that have been proteolytically processed to yield their respective bioactive forms, which may be designated Factor VIIa. Typically, Factor VII is cleaved between residues 152 and 153 to yield Factor VIIa. Such variants of Factor VII may exhibit different properties relative to human Factor VII, including stability, phospholipid binding, altered specific activity, and the like.

As used herein, “Factor VII-related polypeptides” encompasses polypeptides, including variants, in which the Factor VIIa biological activity has been substantially modified or reduced relative to the activity of wild-type Factor VIIa. These polypeptides include, without limitation, Factor VII or Factor VIIa into which specific amino acid sequence alterations have been introduced that modify or disrupt the bioactivity of the polypeptide.

The term “Factor VII derivative” as used herein, is intended to designate wild-type Factor VII, variants of Factor VII exhibiting substantially the same or improved biological activity relative to wild-type Factor VII and Factor VII-related polypeptides, in which one or more of the amino acids of the parent peptide have been chemically modified, e.g. by alkylation, PEGylation, acylation, ester formation or amide formation or the like. This includes but are not limited to PEGylated human Factor VIIa, cysteine-PEGylated human Factor VIIa and variants thereof.

The term “PEGylated human Factor VIIa” means human Factor VIIa, having a PEG molecule conjugated to a human Factor VIIa polypeptide. It is to be understood, that the PEG molecule may be attached to any part of the Factor VIIa polypeptide including any amino acid residue or carbohydrate moiety of the Factor VIIa polypeptide. The term “cysteine-PEGylated human Factor VIIa” means Factor VIIa having a PEG molecule conjugated to a sulfhydryl group of a cysteine introduced in human Factor VIIa.

The biological activity of Factor VIIa in blood clotting derives from its ability to (i) bind to tissue factor (TF) and (ii) catalyze the proteolytic cleavage of Factor IX or Factor X to produce activated Factor IX or X (Factor IXa or Xa, respectively). For purposes of the invention, Factor VIIa biological activity may be quantified by measuring the ability of a preparation to promote blood clotting using Factor VII-deficient plasma and thromboplastin, as described, e.g., in U.S. Pat. No. 5,997,864. In this assay, biological activity is expressed as the reduction in clotting time relative to a control sample and is converted to “Factor VII units” by comparison with a pooled human serum standard containing 1 unit/ml Factor VII activity. Alternatively, Factor VIIa biological activity may be quantified by (i) measuring the ability of Factor VIIa to produce of Factor Xa in a system comprising TF embedded in a lipid membrane and Factor X. (Persson et al., J. Biol. Chem. 272:19919-19924, 1997); (ii) measuring Factor X hydrolysis in an aqueous system; (iii) measuring its physical binding to TF using an instrument based on surface plasmon resonance (Persson, FEBS Letts. 413:359-363, 1997) and (iv) measuring hydrolysis of a synthetic substrate.

Factor VII variants having substantially the same or improved biological activity relative to wild-type Factor VIIa encompass those that exhibit at least about 25%, preferably at least about 50%, more preferably at least about 75% and most preferably at least about 90% of the specific activity of Factor VIIa that has been produced in the same cell type, when tested in one or more of a clotting assay, proteolysis assay, or TF binding assay as described above. Factor VII variants having substantially reduced biological activity relative to wild-type Factor VIIa are those that exhibit less than about 25%, preferably less than about 10%, more preferably less than about 5% and most preferably less than about 1% of the specific activity of wild-type Factor VIIa that has been produced in the same cell type when tested in one or more of a clotting assay, proteolysis assay, or TF binding assay as described above. Factor VII variants having a substantially modified biological activity relative to wild-type Factor VII include, without limitation, Factor VII variants that exhibit TF-independent Factor X proteolytic activity and those that bind TF but do not cleave Factor X.

Variants of Factor VII, whether exhibiting substantially the same or better bioactivity than wild-type Factor VII, or, alternatively, exhibiting substantially modified or reduced bioactivity relative to wild-type Factor VII, include polypeptides having an amino acid sequence that differs from the sequence of wild-type Factor VII by insertion, deletion, or substitution of one or more amino acids.

The terms “Factor VII variant” or “Factor VII variants”, as used herein, is intended to designate Factor VII having the sequence of wild-type factor VII, wherein one or more amino acids of the parent protein have been substituted by another amino acid and/or wherein one or more amino acids of the parent protein have been deleted and/or wherein one or more amino acids have been inserted in protein and/or wherein one or more amino acids have been added to the parent protein. Such addition can take place either at the N-terminal end or at the C-terminal end of the parent protein or both. The “variant” or “variants” within this definition still have FVII activity in its activated form. In one embodiment a variant is 70% identical with the sequence of wild-type Factor VII. In one embodiment a variant is 80% identical with the sequence of wild-type factor VII. In another embodiment a variant is 90% identical with the sequence of wild-type factor VII. In a further embodiment a variant is 95% identical with the sequence of wild-type factor VII.

Growth hormone relevant to present invention includes human growth hormone (hGH), which sequence and characteristics are set forth in, e.g. Hormone Drugs, Gueriguian, U.S.P. Covention, Rockvill, 1982 and growth hormone compounds. The term “growth hormone compound” is intended to indicate human growth hormone (hGH) in which one or more amino acid residues have been deleted and/or replaced by other amino acid residues, natural or unnatural, and/or hGH comprising additional amino acid residues, natural or unnatural, and/or hGH in which at least one organic substituent is bound to one or more organic substituent. Particular mentioning is made of the 191 native amino acid sequence (somatropin) and the 192 amino acid N-terminal methionine species (somatrem).

Examples of cytokines which could be modified according to the present invention include erythropoietin (EPO), thrombopoietin, INF-α, IFN-β, IFN-γ, TNF-α, interleukin-1β (IL-1-β), IL-3, IL-4, IL-5, IL-10, IL-12, IL-15, IL-18, IL-19, IL-20, IL-21 IL-24, grannolyte colony-stimulating factor (G-CSF), GM-CSF, and chemokines such as macrophage inflammatory protein-1 (MIP-1) gamma interferon inducible protein and monokines induced by IFNγ (MIG).

Particular examples of IL-19 relevant to the present invention include those disclosed WO 98/08870 (Human Genome Science), which is incorporated herein by reference.

Particular examples of applicable IL-20 include those disclosed in WO 99/27103 (Zymogenetics), which is incorporated herein by reference. In the present context, IL-20 is intended to indicate IL-20 itself and fragments thereof as well as polypeptides being at least 90% identical to IL-20 or fragments thereof.

Examples of IL-21 relevant to the present invention include those disclosed in WO 00/53761 (Zymogenetics), which is incorporated herein by reference.

TTF is another group of proteins relevant to the present invention. TTF peptides are a family of peptides found mainly in association with the gastrointestinal tract. Particular mentioning is made of breast cancer associated pS2 peptide (TFF-1), which is known from human, mouse, and rat, spasmolytical polypeptide (TFF-2), which is known from human, pig, rat, and mouse and intestinal trefoil factor (TFF-3), known from human, rat and mouse.

Other peptides from the TFF family relevant to the present invention include those disclosed in WO 02/46226 (Novo Nordisk), which is included herein by reference. Other proteins of the TFF family include TFF-1 and TFF-3 dimers as those disclosed in WO 96/06861 (Novo Nordisk), which is incorporated herein by reference.

Several melanorcortin receptors are known, and particular mentioning of peptides applicable for the methods of the present invention is made of peptidic melanocortin-4 receptor agonists, which are known to have an appetite suppressive effect. Particular mentioning is made of peptides or proteins disclosed in the following patent documents, which are all incorporated herein by reference: U.S. Pat. No. 6,054,556 (Hruby), WO 00/05263 (William Harvey Research), WO 00/35952 (Melacure), WO 00/35952 (Melacure), WO 00/58361 (Procter & Gamble), WO 01/52880 (Merck), WO 02/26774 (Procter & Gamble), WO 03/06620 (Palatin), WO 98/27113 (Rudolf Magnus Institute) and WO 99/21571 (Trega).

Other classes of peptides or proteins which are relevant to the present invention include enzymes. Many enzymes are used for various industrial purposes, and particular mentioning is made of hydrolases (proteases, lipases, cellulases, esterases), oxidoreductases (laccases, peroxidaxes, catalases, superoxide dismutases, lipoxygenases), transferases and isomerases.

Other peptides or proteins relevant to the present invention include ACTH, corticotropin-releasing factor, angiotensin, calcitonin, insulin and fragments and analogues thereof, glucagon, IGF-1, IGF-2, enterogastrin, gastrin, tetragastrin, pentagastrin, urogastrin, epidermal growth factor, secretin, nerve growth factor, thyrotropin releasing hormone, somatostatin, growth hormone releasing hormone, somatomedin, parathyroid hormone, thrombopoietin, erythropoietin, hypothalamic releasing factors, prolactin, thyroid stimulating hormones, endorphins, enkephalins, vasopressin, oxytocin, opiods and analogues thereof, asparaginase, arginase, arginine deaminase, adenosine deaminase and ribonuclease.

Insulin is used to treat or prevent diabetes, and in one embodiment, the present invention thus provides a method of treating type 1 or type 2 diabetes, the method comprising administering to a subject in need thereof a therapeutically effective amount of a Hpx fusion protein comprising insulin or an insulin compound according to the present invention. In another embodiment, the invention provides the use of a Hpx fusion protein comprising insulin or an insulin compound according to the present invention in the manufacture of a medicament used in the treatment of type 1 or type 2 diabetes.

GLP-1 may be used in the treatment of hyperglycemia, type 2 diabetes, impaired glucose tolerance, type 1 diabetes, obesity, hypertension, syndrome X, dyslipidemia, β-cell apoptosis, β-cell deficiency, inflammatory bowel syndrome, dyspepsia, cognitive disorders, e.g. cognitive enhancing, neuroprotection, atheroschlerosis, coronary heart disease and other cardiovascular disorders. In one embodiment, the present invention thus provides a method of treating said diseases, the method comprising administering to a subject in need thereof a therapeutically effective amount of a Hpx fusion protein comprising GLP-1 or a GLP-1 compound according to the present invention. In another embodiment, the invention provides the use of a Hpx fusion protein comprising GLP-1 or a GLP-1 compound according to the present invention in the manufacture of a medicament used in the treatment of the above mentioned diseases.

GLP-2 may be used in the treatment of intestinal failure leading to malabsorption of nutrients in the intestines, and in particular GLP-2 may be used in the treatment of small bowel syndrome, Inflammatory bowel syndrome, Crohn's disease, colitis including collagen colitis, radiation colitis, post radiation atrophy, non-tropical (gluten intolerance) and tropical sprue, damaged tissue after vascular obstruction or trauma, tourist diarrhea, dehydration, bacteremia, sepsis, anorexia nervosa, damaged tissue after chemotherapy, premature infants, scleroderma, gastritis including atrophic gastritis, postantrectomy atrophic gastritis and helicobacter pylori gastritis, ulcers, enteritis, cul-de-sac, lymphatic obstruction, vascular disease and graft-versus-host, healing after surgical procedures, post radiation atrophy and chemotherapy, and osteoporosis. In one embodiment the present invention provides methods of treating the above diseases, the method comprising administering to a subject in need thereof a therapeutically effective amount of a Hpx fusion protein comprising GLP-2 or a GLP-2 compound according to this invention. In another embodiment, the present invention provides the use of a Hpx fusion protein comprising GLP-2 or a GLP-2 conjugate according to this invention in the manufacture of a medicament used in the treatment of the above mentioned diseases.

Growth hormone may be used in the treatment of growth hormone deficiency, Turner's syndrome, Prader-Willi Syndrome, chronic renal insufficiency, AIDS wasting and aging. The present invention thus provides a method for treating these diseases or states, the method comprising administering to a patient in need thereof a therapeutically effective amount of a Hpx fusion protein comprising growth hormone or a growth hormone compound according to the present invention. In another embodiment, the invention provides the use of a Hpx fusion protein comprising growth hormone or a growth hormone compound in the manufacture of a medicament used in the treatment of growth hormone deficiency, Turner's syndrome, Prader-Willi Syndrome, chronic renal insufficiency, AIDS wasting or aging.

Cytokines are implicated in the etiology of a host of diseases involving the immune system. In particular it is mentioned that IL-20 is involved in psoriasis and its treatment, and that IL-21 is involved in cancer and could constitute a treatment to this disease. In one embodiment, the invention provides a method for the treatment of psoriasis comprising the administration of a Hpx fusion protein comprising an IL-20 conjugate according to the present invention. In another embodiment, the invention relates to the use of a Hpx fusion protein comprising an IL-20 conjugate of the present invention in the manufacture of a medicament used in the treatment of psoriasis.

In another embodiment, the present invention relates to a method of treating cancer, the method comprising administration of a Hpx fusion protein comprising an IL-21 conjugate of the present invention to a subject in need thereof. In another embodiment, the invention relates to the use of a Hpx fusion protein comprising an IL-21 conjugate according to the present invention in the manufacture of a medicament used in the treatment of cancer.

TTF proteins may be used to increase the viscosity of mucus layers in subject, to reduce secretion of salvia, e.g. where the increase salvia secretion is caused by irradiation therapy, treatment with anticholinergics or Sjögren's syndrome, to treat allergic rhinitis, stress induced gastric ulcers secondary to trauma, shock, large operations, renal or liver diseases, treatment with NSAID, e.g. aspirin, steroids or alcohol. TTF peptides may also be used to treat Crohn's disease, ulcerative colitis, keratoconjunctivitis, chronic bladder infections, intestinal cystitis, papillomas and bladder cancer. In one embodiment, the invention thus relates the a method of treating the above mention diseases or states, the method comprising administering to a subject patient in need thereof a therapeutically effective amount of Hpx fusion protein comprising TTF according to the present invention. In another embodiment, the invention relates the use of Hpx fusion protein comprising TTF of the present invention in the manufacture of a medicament for the treatment of the above mentioned diseases or states.

Melanocortin receptor modifiers, and in particular melanorcortin 4 receptor agonists have been implicated the treatment and prevention of obesity and related diseases. In one embodiment, the present invention provides a method for preventing or delaying the progression of impaired glucose tolerance (IGT) to non-insulin requiring type 2 diabetes, for preventing or delaying the progression of non-insulin requiring type 2 diabetes to insulin requiring diabetes, for treating obesity and for regulating the appetite. Melanocortin 4 receptor agonists have also been implicated in the treatment of diseases selected from atherosclerosis, hypertension, diabetes, type 2 diabetes, impaired glucose tolerance (IGT), dyslipidemia, coronary heart disease, gallbladder disease, gall stone, osteoarthritis, cancer, sexual dysfunction and the risk of premature death. In one embodiment, the invention thus provides a method of treating the above diseases or states, the method comprising administering to a subject in need thereof a therapeutically effective amount of a Hpx fusion protein comprising a melanocortin 4 receptor agonist of the present invention. In still another embodiment, the invention relates to the use of a Hpx fusion protein comprising a melanocortin 4 receptor agonist of the present invention in the manufacture of a medicament for the treatment of the above mentioned diseases or states.

Factor VII compounds have been implicated in the treatment of disease related to coagulation, and biological active Factor VII compounds in particular have been implicated in the treatment of hemophiliacs, hemophiliacs with inhibitors to Factor VIII and IX, patients with thrombocytopenia, patients with thrombocytopathies, such as Glanzmann's thrombastenia platelet release defect and storage pool defects, patient with von Willebrand's disease, patients with liver disease and bleeding problems associated with traumas or surgery. Biologically inactive Factor VII compounds have been implicated in the treatment of patients being in hypercoagulable states, such as patients with sepsis, deep-vein thrombosis, patients in risk of myocardial infections or thrombotic stroke, pulmonary embolism, patients with acute coronary syndromes, patients undergoing coronary cardiac, prevention of cardiac events and restenosis for patient receiving angioplasty, patient with peripheral vascular diseases, and acute respiratory distress syndrome. In one embodiment, the invention thus provides a method for the treatment of the above mentioned diseases or states, the method comprising administering to a subject in need thereof a therapeutically effective amount of a Hpx fusion protein comprising a Factor VII compound according to the present invention. In another embodiment, the invention provides the use of a Hpx fusion protein comprising a Factor VII compound according to the present invention in the manufacture of a medicament used in the treatment of the above mentioned diseases or states.

Many diseases are treated using more than one medicament in the treatment, either concomitantly administered or sequentially administered. It is therefore within the scope of the present invention to use the peptide conjugates of the present invention in therapeutic methods for the treatment of one of the above mentioned diseases in combination with one or more other therapeutically active compound normally used to in the treatment said disease. By analogy, it is also within the scope of the present invention to use the peptide conjugates of the present invention in combination with other therapeutically active compounds normally used in the treatment of one of the above mentioned diseases in the manufacture of a medicament for said disease.

The above therapeutic methods may comprising administration via any suitable route, such as the oral, rectal, nasal, pulmonary, topical (including buccal, sublingual), transdermal, intracisternal, intraperitoneal, vaginal, parenteral (including subcutaneous, intramuscular, intrathecal, intravenous and intradermal) route, the parenteral route being preferred.

A typical parenteral dose is in the range of 10⁻⁹ mg/kg to about 100 mg/kg body weight per administration. Typical administration doses are from about 0.0000001 to about 10 mg/kg body weight per administration. The exact dose will depend on e.g. indication, medicament, frequency and mode of administration, the sex, age and general condition of the subject to be treated, the nature and the severity of the disease or condition to be treated, the desired effect of the treatment and other factors evident to the person skilled in the art.

Typical dosing frequencies are twice daily, once daily, bi-daily, twice weekly, once weekly or with even longer dosing intervals. Due to the prolonged half-lifes of the fusion proteins of the present invention, a dosing regime with long dosing intervals, such as twice weekly, once weekly or with even longer dosing intervals is a particular embodiment of the invention.

### Pharmaceutical Compositions

Another object of the present invention is to provide a pharmaceutical formulation comprising a Hpx fusion protein compound which is present in a concentration from 10⁻¹⁵ mg/ml to 200 mg/ml, such as e.g. 10⁻¹⁰ mg/ml to 5 mg/ml and wherein said formulation has a pH from 2.0 to 10.0. The formulation may further comprise a buffer system, preservative(s), tonicity agent(s), chelating agent(s), stabilizers and surfactants. In one embodiment of the invention the pharmaceutical formulation is an aqueous formulation, i.e. formulation comprising water. Such formulation is typically a solution or a suspension. In a further embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical formulation is a freeze-dried formulation, whereto the physician or the patient adds solvents and/or diluents prior to use.

In another embodiment the pharmaceutical formulation is a dried formulation (e.g. freeze-dried or spray-dried) ready for use without any prior dissolution.

In a further aspect the invention relates to a pharmaceutical formulation comprising an aqueous solution of a Hpx fusion protein, and a buffer, wherein said Hpx protein is present in a concentration from 0.1-100 mg/ml or above, and wherein said formulation has a pH from about 2.0 to about 10.0.

In a another embodiment of the invention the pH of the formulation is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0.

In a further embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the formulation further comprises a pharmaceutically acceptable preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 20^{th} edition, 2000.

In a further embodiment of the invention the formulation further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects obtained using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 20^{th} edition, 2000.

In a further embodiment of the invention the formulation further comprises a chelating agent. In a further embodiment of the invention the chelating agent is selected from salts of ethylenediaminetetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 20^{th} edition, 2000.

In a further embodiment of the invention the formulation further comprises a stabilizer. The use of a stabilizer in pharmaceutical compositions is well-known to the skilled person. For convenience reference is made to Remington: The Science and Practice of Pharmacy, 20^{th} edition, 2000.

More particularly, compositions of the invention are stabilized liquid pharmaceutical compositions whose therapeutically active components include a protein that possibly exhibits aggregate formation during storage in liquid pharmaceutical formulations. By “aggregate formation” is intended a physical interaction between the protein molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a protein during storage of a liquid pharmaceutical composition can adversely affect biological activity of that protein, resulting in loss of therapeutic efficacy of the pharmaceutical composition. Furthermore, aggregate formation may cause other problems such as blockage of tubing, membranes, or pumps when the protein-containing pharmaceutical composition is administered using an infusion system.

The pharmaceutical compositions of the invention may further comprise an amount of an amino acid base sufficient to decrease aggregate formation by the protein during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L, D, or mixtures thereof) of a particular amino acid (methionine, histidine, arginine, lysine, isoleucine, aspartic acid, tryptophan, threonine and mixtures thereof) or combinations of these stereoisomers or glycine or an organic base such as but not limited to imidazole, may be present in the pharmaceutical compositions of the invention so long as the particular amino acid or organic base is present either in its free base form or its salt form. In one embodiment the L-stereoisomer of an amino acid is used. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the protein during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphuric amino acids or amino acid analogous) may be added to inhibit oxidation of methionine residues to methionine sulfoxide when the protein acting as the therapeutic agent is a protein comprising at least one methionine residue susceptible to such oxidation. By “inhibit” is intended minimal accumulation of methionine oxidized species over time. Inhibiting methionine oxidation results in greater retention of the protein in its proper molecular form. Any stereoisomer of methionine (L or D isomer) or any combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be obtained by adding methionine such that the ratio of methionine added to methionine residues ranges from about 1:1 to about 1000:1, such as 10:1 to about 100:1.

In a further embodiment of the invention the formulation further comprises a stabilizer selected from the group of high molecular weight polymers or low molecular compounds. In a further embodiment of the invention the stabilizer is selected from polyethylene glycol (e.g. PEG 3350), polyvinyl alcohol (PVA), polyvinylpyrrolidone, carboxy/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active protein therein. Stabilizing agents of particular interest to the present invention include, but are not limited to, methionine and EDTA, which protect the protein against methionine oxidation, and a nonionic surfactant, which protects the protein against aggregation associated with freeze-thawing or mechanical shearing.

In a further embodiment of the invention the formulation further comprises a surfactant. In a further embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (e.g. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (e.g. phosphatidyl serine, phosphatidyl choline, phosphatidyl ethanolamine, phosphatidyl inositol, diphosphatidyl glycerol and sphingomyelin), derivates of phospholipids (e.g. dipalmitoyl phosphatidic acid) and lysophospholipids (e.g. palmitoyl lysophosphatidyl-L-serine and 1-acyl-sn-glycero-3-phosphate esters of ethanolamine, choline, serine or threonine) and alkyl, alkoxyl(alkyl ester), alkoxy(alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (e.g. cephalins), glyceroglycolipids (e.g. galactopyransoide), sphingoglycolipids (e.g. ceramides, gangliosides), dodecylphosphocholine, hen egg lysolecithin, fusidic acid derivatives—(e.g. sodium tauro-dihydrofusidate etc.), long-chain fatty acids and salts thereof C₆-C₁₂ (e.g. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (e.g. Dodecyl β-D-glucopyranoside), poloxamines (e.g. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 20^{th} edition, 2000.

It is possible that other ingredients may be present in the pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing a Hpx fusion protein according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the Hpx fusion protein, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the formulation of solids, semisolids, powder and solutions for pulmonary administration of Hpx fusion protein, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the Hpx fusion protein in the form of a nasal or pulmonal spray. As a still further option, the pharmaceutical compositions containing the Hpx fusion protein of the invention can also be adapted to transdermal administration, e.g. by needle-free injection or from a patch, optionally an iontophoretic patch, or transmucosal, e.g. buccal, administration.

The term “stabilized formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person. Physical stability of the aqueous protein formulations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as anthracene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

In one embodiment of the invention the pharmaceutical formulation comprising the Hpx fusion protein is stable for more than 6 weeks of usage and for more than 3 years of storage.

In another embodiment of the invention the pharmaceutical formulation comprising the Hpx fusion protein is stable for more than 4 weeks of usage and for more than 3 years of storage.

In a further embodiment of the invention the pharmaceutical formulation comprising the Hpx fusion proteinis stable for more than 4 weeks of usage and for more than two years of storage.

In an even further embodiment of the invention the pharmaceutical formulation comprising the Hpx fusion protein is stable for more than 2 weeks of usage and for more than two years of storage.

### EXAMPLES

### Abbreviations

NDSB: Non-detergent sulfo betaine
NDSB201: 3-(1-pyridino)-1-propane solfonate

### DTT: Dithiothreitol

IPTG: Isopropyl-β-D-thiogalacto pyranoside

### Example 1

### Hemopexin Cloning

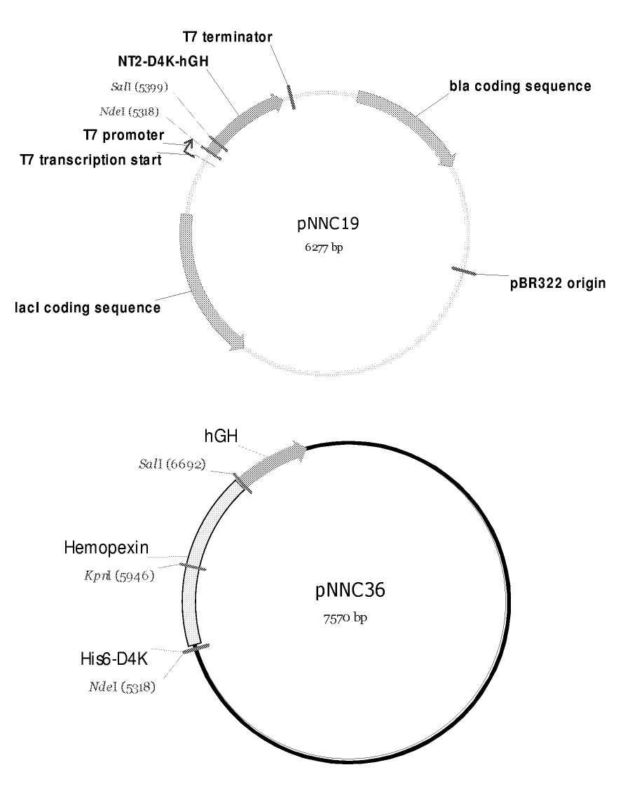

Four hemopexin protein sequences were found in the NCBI protein database (gi: 1708182; 2144904; 226337; 386789). These where aligned and found to be identical using Vector NTI software. A corresponding cDNA sequence representing linear mRNA (gi: 11321560) was used as template for primer design. The primers were designed to exclude the signal peptide (see below). A nested primer PCR strategy was utilized with a short Hpx-specific primer set to amplify Hpx from oligo (dT) primed double stranded human liver cDNA (BD Bioscience), and a longer primer set containing an additional purification tag (His6), an enterokinase cleavage site (D4K), a short hGH linker sequence, as well as Nde1 and Sal1 restriction sites for cloning into a bacterial expression vector (pNNC19) already containing the hGH gene (see FIG. 1). High fidelity DNA polymerase was used for 25 PCR cycles with the short primer set and the identity of the amplicon confirmed by gel electrophoresis (amplicon size 1317 bp) and restriction enzyme digestion of the Kpn1 site in Hpx. After this the purified amplicon was submitted to another 10 cycles using the longer primer set. The amplicon size was 1394 bp.

### Hemopexin Short Primer Set:

| | |
|---|---|
| 5′-acccctcttcctccgactagtgccca-3′ | (SEQ ID No 1) |
| and | |
| 5′-gtgagtgcagcccaggagactggtca-3′. | (SEQ ID No 2) |

### Hemopexin Long Primer Set (Containing Purification Tag, Nde1 and Sal1 Restriction Sites):

(SEQ ID No 3)

5′agatatacatatgcaccatcaccatcatcacgacgatgacgacaagac
ccctcttcctccgactagtgccca-3′
and

(SEQ ID No 4)

5′caaaaagtcgactcagcgggatggtcgggaagtgagtgcagcccagga
gactggt-3′.

### Example 2

### Hpx-hGH Cloning

The ends of the amplicon obtained in Example 1 were cut with Nde1 and Sal1 and the fragment cloned in pNNC19. Positive clones were selected using restriction enzyme analysis and the sequence of the fusion protein confirmed by DNA sequencing. The resulting expression vector encodes a fusion protein containing Met-His6-D4K-Hpx-hGH (see FIG. 1).

### Example 3

### Expression and Isolation of His6-D4K-Hpx-hGH

### Expression

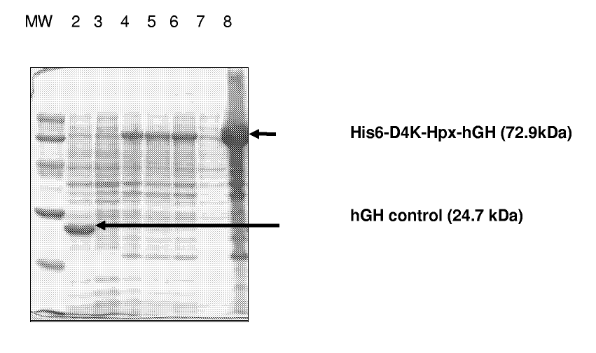

Expression was performed at standard conditions using different temperatures and concentrations of IPTG. Briefly, E. coli bacteria were grown to an OD_{600 nm} of 0.6 and expression was induced with IPTG for approximately 4 hours. The bacteria pellet was harvested and the protein content investigated for the presence of recombinant fusion protein using SDS PAGE (see FIG. 2). The Hpx fusion protein was found mainly in inclusion bodies in all conditions tested.

### Refolding and Purification

After solubilisation of His6-D4K-Hpx-hGH inclusion bodies (in 8 M Urea, 20 mM DTT, 20 mM Tris pH 9.0), the protein was refolded by 50-fold dilution refolding in 1 M NDSB201, 1 M Urea, and 20 mM Tris pH 9.0 over night. Correct refolding of the hemopexin part of the fusion protein was shown by its ability to bind hemin. Hemin binding is dependent on the coordinated binding of histidine 213 and 270 to the heme iron forming a stable bis-histidyl Fe(III) complex, something that only can be achieved when hemopexin is folded in its natural conformation. Furthermore, free hemin absorbs at 390 nm and at 405 nm when bound to hemopexin in the bis-histidyl Fe(III) complex (see Morgan W T and Muller-Eberhard U (1972), J. Biol. Chem., 247:7181-7187 and Heide K et al. (1964) Clin. Chim. Acta., 10:460-469). Proteins in general absorbs at 280 nm. Hemin/Hpx-hGH complexes can thus be specifically followed during purification by monitoring UV 280/390 nm.

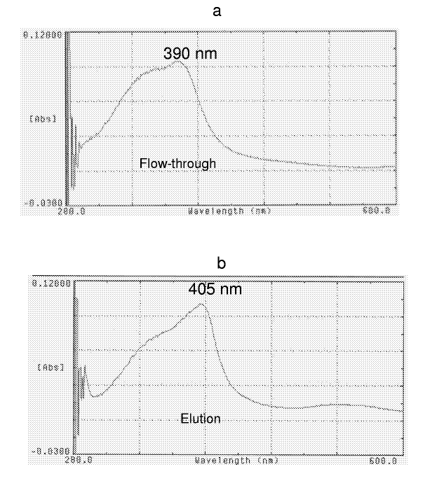

During purification of refolded Hpx-hGH, hemin was added in molar excess and absorbance at 390 nm was seen in the flow through and in the eluting peak. However, as evident by the UV scans in FIGS. 3a and b, the absorbance profile changes from the flow through to the elution, which indicates that free hemin is found in the flow through (peak at 390 nm) and bound hemin is found in the eluting (peak at 405 nm). The biological activity of the hGH domain can be tested in an assay suitable for assessing the biological activity of hGH, one example of which is the BAF 3 assay (Wada M et al. (1997), Mol Endocrinol 12:146-156).

In addition, after refolding of the protein, disulphide bridge mapping was performed by in-gel trypsin digest followed by MALDI analysis before and after DTT treatment of the trypsin fragments. Hpx-hGH contains six disulphide bridges in the Hpx part (Cys27-Cys208, Cys126-Cys131, Cys165-Cys177, Cys234-Cys437, Cys343-Cys385, Cys395-Cys412) and two disulphide bridges in the hGH part of the protein (Cys53-Cys165, Cys182-Cys189). Formation of four out of eight disulphide bridges in the fusion protein could be identified; two in the Hpx part (Cys165-Cys177 and Cys395-Cys412) and two in the hGH part (Cys53-Cys165 and Cys182-Cys189) suggesting that the Hpx domain forms disulphide bridging correctly and verifying that the hGH domain forms the correct disulphide bridges. It is quite common that not all disulphide bridges can be detected in MALDI analysis. Taken together, the ability of the Hpx domain in Hpx-hGH to bind hemin, and the positive identification of two out of six disulphide bridges, strongly indicate that the Hpx domain is folded correctly. In addition, positive identification of both disulphide bridges in the hGH domain also imply that this domain is folded correctly. Thus, Hpx-hGH fusion protein with the functional domains intact seems to be successfully obtained.

As the present fusion protein comprises a H is purification tag it may conveniently be purified on a nickel affinity column. Refolded His6-D4K-Hpx-hGH was loaded onto Ni-NTA Agarose (Qiagen). However, the Ni-ions are stripped from the matrix during purification, which is believed to be caused by Ni-binding to Hpx. The column was run several times using different buffers but nothing prevented the nickel strip from the column.

As an alternative strategy, the fusion protein may be purified on an anion exchange column, e.g. Q Sepharose FF, in which case the histidine purification tag is redundant.

Treatment of the histidine tag containing fusion protein with enterokinase will result in Hpx-hGH fusion protein (SEQ ID No 5).

1. A fusion protein comprising a first protein fused to Hemopexin (Hpx) or a Hpx variant, optionally via a linker.

2. The fusion protein according to claim 1, wherein said fusion protein has an increased circulation time compared to said first protein.

3. The fusion protein according to claim 1, wherein said first protein is a therapeutic protein.

4. The fusion protein according to claim 1 wherein said Hpx variant is Hpx₁₋ₙ, wherein n is an integer from 360 to 438.

5. The fusion protein according to claim 1, wherein said Hpx variant is Hpxₘ₋₄₃₉, wherein m is an integer from 2 to 100.

6. The fusion protein according to claim 1, wherein said Hpx variant is Hpx_{q-p}, wherein q is an integer from 2-50, and p is an integer from 389 to 438.

7. The fusion protein according to claim 1, wherein said Hpx variant is obtained by adding, deleting and/or substituting up to 50 amino acid residues from Hpx.

8. The fusion protein according to claim 1, wherein said Hpx variant has at least 80% sequence identity to Hpx.

9. The fusion protein according to claim 1, wherein fusion protein has a molecular weight above 65 kDa.

10. The fusion protein according to claim 1, wherein said Hpx variant comprises a deletion or substitution of one or more of His 213 and His 270 in the Hpx sequence.

11. (canceled)

12. The fusion protein according to claim 1, wherein a linker is present.

13. The fusion protein according to claim 1, wherein the N-terminal of said first protein is fused, optionally via linker, to the C-terminal of Hpx.

14. The fusion protein according to claim 1, wherein the C-terminal of said first protein is fused, optionally via linker, to the N-terminal of Hpx.

15. The fusion protein according to claim 1, wherein said first protein is a growth hormone, a growth hormone compound or human growth hormone.

16. A fusion protein according to claim 15 selected from the group consisting of

| | |
|---|---|
| Hpx-hGH; | (SEQ ID No: 5) |
| hGH-Hpx; | (SEQ ID No: 6) |
| Hpx(H213A)-hGH; | (SEQ ID No: 7) |
| hGH-Hpx(H213A); | (SEQ ID No: 8) |
| His6-D4K-Hpx-hGH; | (SEQ ID No: 9) |
| and | |
| His8-PTERP-D4K-hGH-Hpx. | (SEQ ID No: 10) |

17. A Hpx variant wherein said variant is Hpx₁₋ₙ, wherein n is an integer from 360 to 438.

18. A Hpx variant wherein said variant is Hpxₘ₋₄₃₉, wherein m is an integer from 2 to 100.

19. A Hpx variant wherein said variant is Hpx_{q-p}, wherein q is an integer from 2-50, and p is an integer from 389 to 438.

20. A Hpx variant wherein said variant comprises a deletion or substitution of up to 50 amino acid residues from Hpx.

21. A Hpx variant which has at least 80% sequence identity to Hpx.

22. A Hpx variant comprising a deletion or substitution of one or more of His 213 and His 270 in the Hpx sequence.

23. A method of increasing circulation time of a first protein, the method comprising preparing a fusion protein between (i) said first protein and (ii) Hpx or a Hpx variant, optionally via a linker.

24-32. (canceled)

33. The method according to claim 23, which comprises refolding the fusion protein by dilution refolding in an aqueous non-detergent sulfobetaine and urea at a pH above 7.

34. (canceled)

35. A nucleic acid construct comprising a nucleic acid sequence encoding a protein according to claim 1.

36. A vector comprising the nucleic acid construct according to claim 35.

37. A host cell comprising the vector according to claim 36.

38. (canceled)

39. A pharmaceutical composition comprising a fusion protein according to claim 1

40. A method of treating growth hormone deficiency, Turner's syndrome, Prader-Willi Syndrome, chronic renal insufficiency, AIDS wasting or aging, the method comprising administering to a patient in need thereof an effective amount of an Hpx fusion protein according to claim 15.

41-43. (canceled)

44. An antibody that specifically binds to a Hpx fusion protein according to claim 1.
